(19) United States
(12) Patent Application Publication
(10) Pub. No.: US 2009/0082729 A1
(43) Pub. Date: Mar. 26, 2009

(54) Personal cleaning device

(76) Inventor: Stephen Callow, Toronto (CA)

Correspondence Address: BERESKIN AND PARR, 40 KING STREET WEST, BOX 401, TORONTO, ON, M5H 3Y2, CA

(21) Appl. No.: 11/902,816

(22) Filed: Sep. 26, 2007

(51) Int. Cl. A61M 31/00

(52) U.S. Cl. 604/150; 604/257

(57) ABSTRACT

A douche device which can be supported on a shower head. The device has a reservoir, a cross-support which can be removably connected to the reservoir, and a central semi-circular portion in the cross-support which can be clipped to the shower head pipe. The reservoir has a bottom outlet connected by a flexible hose to a douche wand. The reservoir also has an overflow outlet located on its rear wall above the bottom outlet, with an unobtrusive flexible overflow hose extending from the overflow outlet down to the floor. In use, the device can readily be connected to or disconnected from a shower head, and when in use, excess water from the shower head will not overfill the reservoir and fall onto the user from above. Instead, excess water will exit the reservoir via the overflow opening and will be conducted down to the floor and into a floor drain.

### FIELD OF THE INVENTION

This invention relates to a personal cleaning device for directing a flow of water into body orifices. It particularly relates to douches.

### BACKGROUND OF THE INVENTION

Douches are commonly needed and are frequently used, but ordinary bathrooms which contain a bathtub and/or a shower stall are not usually equipped with a douche. In addition, the person wishing to use the douche may prefer not to wet any more of her body than necessary, thus making an ordinary shower or bathtub unsuitable for this purpose.

Devices have been created in the past for attachment, for example to a shower, for providing a douche. A typical example of this type of device is shown in U.S. Pat. No. 3,162,193 issued to F. B. Zacks on Dec. 22, 1964. In the Zacks device, a reservoir is hung on a shower head, with a tube from the reservoir extending to a douche nozzle. A disadvantage of this device is that if water from the shower head is run too slowly, the reservoir will empty and the douche will cease functioning. Conversely, if water from the shower head is run too hard, the reservoir will quickly overfill and the excess water from it will fall on the user, which is usually undesirable. In addition the pressure from the douche nozzle will vary depending on the water level in the reservoir.

### SUMMARY OF THE INVENTION

It is therefore an object of this invention to provide an improved douche device which can for example be supported on the pipe which carries the shower head, and in which there is no need to match the water flow from the shower head to the water flow of the douche device. If excess water flows from the shower head, it will flow down a separate overflow tube and will flow to the drain, and will not spray on the user.

In one aspect the invention provides a douche device adapted to be supported on a water spray fitting of a bath or a shower, said spray fitting being of the kind having a spray head and a connecting portion connecting said spray head to a wall, said douche device comprising: a reservoir, a support for removably supporting said reservoir on said spray fitting with the spray head of said spray fitting located within said reservoir, said reservoir having a lower first opening, an outlet hose having one end connected to said first opening and having a second end, a wand connected to said second end and having a plurality of openings therein for spraying water on a user, said reservoir having a second opening located above said first opening, and an overflow hose connected to said second opening, so that in use, water from said spray fitting may fill said reservoir to the level of said second opening, and so that water in said reservoir can be sprayed from said wand onto a user, and so that excess water from the spray fitting will not overflow the reservoir and fall onto a user but instead will leave the reservoir via the overflow hose.

Further aspects and advantages of the invention will appear from the following description, together with the accompanying drawings.

### BRIEF DESCRIPTION OF THE DRAWINGS

In the drawings:

FIG. 1 is a perspective exploded view of a douche device according to the invention ready to be connected to a shower head;

FIG. 2 is a perspective view of the douche device of FIG. 1, connected to a shower head; and

FIG. 3 is a side sectional view of the douche device of FIG. 1, connected to a shower head.

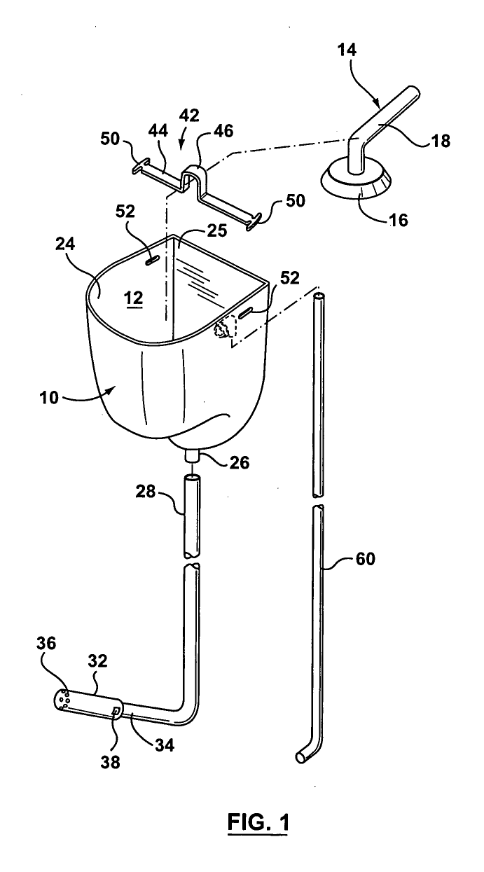

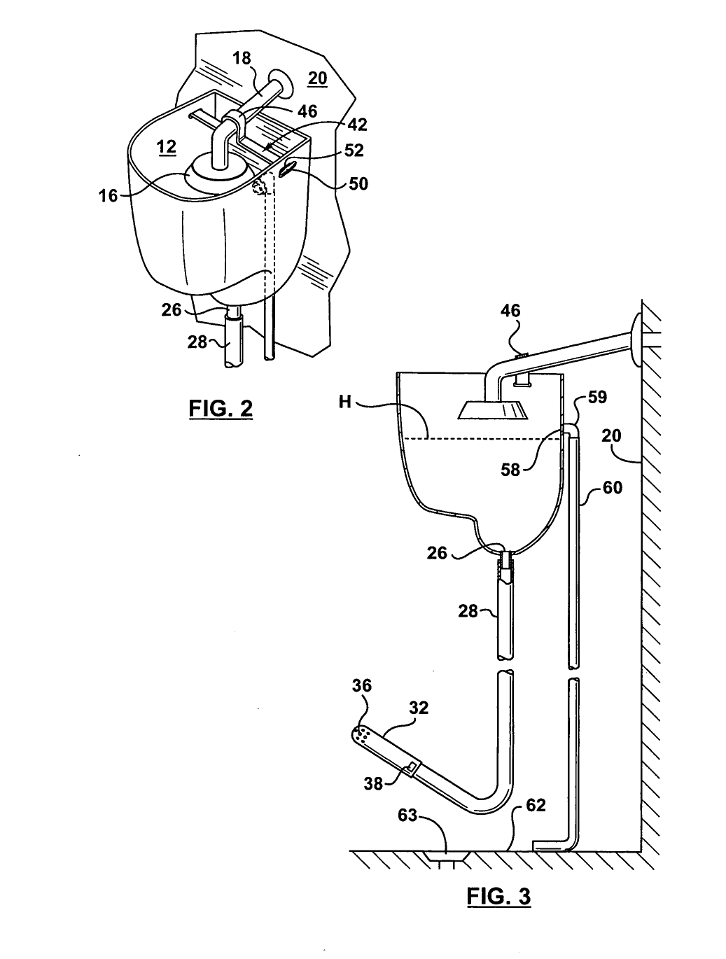

### DETAILED DESCRIPTION OF THE INVENTION

As shown in the drawings, a typical douche device according to the invention includes a reservoir 10 made of any suitable elastomeric material. A preferred material is a rubber/silicone compound sold by Dow Corning of Michigan, U.S.A. under the trademark “T-BLEND”, which is injection molded to form the reservoir. Other materials may also be used, e.g. polyethylene, polypropylene, polyurethane or the like. The reservoir 10 may assume a variety of shapes but is large enough to define an interior volume 12 which accommodates a water spray fitting 14. The fitting 14 includes a shower head 16 connected by a pipe 18 to a wall 20. The shower head 16 is normally located high on the wall 20, at a position above the user, so that when the douche device is not present, the shower head 16 can spray water on the user from a position above the user.

The reservoir 10 includes a sidewall 24 enclosing the volume 12 (except for the top, which is open), so that the reservoir 10 as viewed from the side or front is generally of a funnel shape. The wall thickness of the reservoir 10 should be sufficient so that the reservoir will retain its shape and will not droop and spill water when it is filled with water.

The reservoir sidewall 24 includes a flat rear wall 25. At the bottom of the reservoir 10, an open-ended outlet tube 26 integral with the reservoir projects downwardly a short distance and ends at a connection to one end of a flexible outlet hose 28. The outlet hose 28 is normally connected to the outlet tube 26 simply by being pushed over the end of the outlet tube 26. The outlet tube 26, and hose 28 can be glued together, or a separate circular clamp (not shown) may be used to secure them together, or a conventional barbed connector (not shown) may be used.

A conventional water applicator wand 32 is connected in any desired manner to the other end 34 of the flexible outlet hose 28. As is well-known, the water applicator wand 32 is perforated, containing a number of small holes 36 on its tip from which water sprays forwardly, and includes a button valve 38 which can be pressed by the user to turn the water flow on and off.

The reservoir 10 is supported on the pipe 18 of the shower head 16 by a plastic cross-member 42. The plastic cross-member 42 is made of relatively rigid plastic with some “springiness” and has the form of an essentially flat cross-strip 44 with a central curved section 46. The curved section 46 has the form of a semi-circle and is of diameter slightly less than that of the shower head pipe 18, so that the curved section 46 can be snapped over the shower head pipe. The cross-member 42 typically also includes small enlargements 50 one at each end thereof. The enlargements 50 are dimensioned so that each can be fitted through a small slot 52, one at each side of the side wall 24. The slots 52 are located near the rear wall 25 but are positioned so that when the reservoir 10 is filled with water, there will be an approximately equal weight of water in front of and behind the slots 52. This allows the reservoir 10 to hang from pipe 18 in an even, balanced condition.

For installation of the douche device, the curved section 46 of the cross-member 42 is snapped onto the shower head pipe 18. The reservoir 10 is then moved so that the enlargements 50 at the ends of the cross-member 42 are pushed through the slots 52, one at each side of the reservoir 10. The reservoir 10 is now held and supported in position on the pipe 18.

In the rear wall 25 of the reservoir 10, well above the bottom of the outlet tube 26 but below the open top 54 of the reservoir, there is located an overflow opening 58. An L-shaped overflow elbow 59 is connected to the overflow opening 58, and a flexible overflow hose 60, which is preferably of a flattened shape and of transparent or translucent plastic, is connected to the overflow elbow 59. The overflow hose 60 extends down to the floor 62, to allow excess water to exit from the reservoir and flow into the tub or shower drain 63. (If desired, the overflow hose 60 may be of a solid color instead of being transparent or translucent.)

In use, the user turns on the tap or taps which control the flow of water from the shower head 16 and allows the reservoir 10 to fill to the level of the overflow opening 58. At this point, water will run through the holes 36 in the wand 32 if the button valve 38 is pressed. If the water flow from the shower head 16 is sufficiently strong, there will be excess water which will run down through the overflow hose 60 and into the drain. The user adjusts the temperature of either of these two flows of water and can then use the wand. To prevent the reservoir 10 from running dry during use, normally some excess flow is provided from the shower head 16, and the excess simply exits via the overflow hose 60, without falling on the user.

It will be seen then even if the flow from the shower head 16 varies, the hydrostatic pressure of water entering the wand 32 depends solely on the height (H) of the water level in the reservoir 10 above the wand 32. There is no variation of the height (H) with variations in the water flow from the shower head 16, so long as the flow from shower head 16 exceeds the flow from the wand 32.

In addition, because of the presence of the overflow opening 58 and the overflow hose 60, the excess water from the shower head 16 does not overfill the reservoir 10 and does not fall on to the user (although it may wet his/her feet). The overflow opening 58, elbow 59, and hose 60, are preferably made sufficiently large to accommodate as high an excess flow as the shower head 16 is likely to deliver under normal use conditions.

When the reservoir 10 is injection molded from the very flexible “T-BLEND” (trademark Dow Corning) rubber/silicone compound mentioned, or from another flexible resilient plastic material, then the reservoir 10 can be folded to occupy a very small volume (for travel) and can be secured in its folded position by simple securing means such as an elastic band, a Velcro strip, or by a carrying bag. Similarly, the outlet hose and the overflow hose are of flexible material and can be removed and separately folded. This allows the device to occupy minimal space in a suitcase so that it can be taken on trips by a traveler.

When the constraints (e.g. an elastic band or a Velcro strip) are removed from the folded reservoir 10, it then springs into its full open shape, so that it can be installed as described previously.

While preferred embodiments of the invention have been described, it will be realized that various changes can be made within the scope of the invention.

1. A douche device adapted to be supported on a water spray fitting of a bath or a shower, said spray fitting being of the kind having a spray head and a connecting portion connecting said spray head to a wall, said douche device comprising: a reservoir, a support for removably supporting said reservoir on said spray fitting with the spray head of said spray fitting located within said reservoir, said reservoir having a lower first opening, an outlet hose having one end connected to said first opening and having a second end, a wand connected to said second end and having a plurality of openings therein for spraying water on a user, said reservoir having a second opening located above said first opening, and an overflow hose connected to said second opening, so that in use, water from said spray fitting may fill said reservoir to the level of said second opening, and so that water in said reservoir can be sprayed from said wand onto a user, and so that excess water from the spray fitting will not overflow the reservoir and fall onto a user but instead will leave the reservoir via the overflow hose.

2. Apparatus according to claim 1 wherein said support includes a cross-member having a generally semi-circular portion adapted to be clipped onto said connecting portion, said connecting portion being a pipe.

3. Apparatus according to claim 2 wherein said reservoir has a sidewall having a pair of slots therein, and said cross-member has enlarged ends sized to be inserted through said slots in said sidewall for said cross-member to retain said reservoir on said cross-member.

4. Apparatus according to claim 1 wherein said reservoir has a rear wall, and wherein said second opening is located in said rear wall.

5. Apparatus according to claim 4 wherein said overflow hose is a generally flat, thin tube.

6. Apparatus according to claim 1 wherein said connecting portion, said outlet hose and said overflow hose, are removably connected to said reservoir, and wherein said reservoir is made of a flexible plastic, so that said reservoir, after said connecting portion and said hoses are disconnected from it, can be folded to facilitate traveling with said device.

7. Apparatus according to claim 6 wherein said plastic of said reservoir is resilient, so that when said reservoir is secured in a folded condition by a constraint, and when said constraint is removed, said reservoir will itself unfold into a substantially open condition.
